(19) United States
(12) Patent Application Publication
(10) Pub. No.: US 2024/0082049 A1
(43) Pub. Date: Mar. 14, 2024

(54) METHOD FOR CONTROLLING A FLEXIBLE THERMOELECTRIC ELEMENT AND DYNAMIC THERMAL THERAPY DEVICE USING THE SAME

(71) Applicant: TEGWAY CO., LTD, Daejeon (KR)

(72) Inventors: Kyoung Soo YI, Daejeon (KR); Ock Kyun OH, Daejeon (KR); Se Hwan YIM, Daejeon (KR); Jin Seong NOH, Daejeon (KR); Se Hong CHEON, Daejeon (KR)

(73) Assignee: TEGWAY CO., LTD, Daejeon (KR)

(21) Appl. No.: 18/273,743

(22) PCT Filed: Jan. 11, 2022
(86) PCT No.: PCT/KR2022/000445

§ 371 Date: Jul. 21, 2023

(30) Foreign Application Priority Data
Jan. 22, 2021 (KR) 10-2021-0009555

(51) Int. Cl. A61F 7/00

(52) U.S. Cl. CPC A61F 7/007; A61F 2007/0075; A61F 2007/0093; A61F 2007/0282

(57) ABSTRACT

The present invention relates to a method of controlling a flexible thermoelectric element to provide dynamic thermal therapy, and to a dynamic thermal therapy device, wherein the flexible thermoelectric element has a plurality of thermoelectric modules, each corresponding to a plurality of regions arranged sequentially, wherein the plurality of thermoelectric modules can selectively perform either exothermic or endothermic operation on the plurality of regions by control of a current.

### TECHNICAL FIELD

The present invention relates to a method for controlling a flexible thermoelectric element that can be utilized for skin care, and a dynamic thermal therapy device using the same.

### BACKGROUND ART

A thermoelectric device is a device that directly converts temperature differences into electrical energy, using a phenomenon called the Seebeck effect, or the Peltier effect, which allows the same device to act as a heater or cooler by running a current through it.

Thermoelectric devices have a wide range of applications. They are used to measure and control temperatures in scientific laboratories and factories, especially in furnaces and other inaccessible or dangerous areas. Thermoelectric devices can be used in remote-controlled equipment such as telephone repeaters, weather stations in the Arctic, and navigational buoys, that require moderate power.

Today, the scope of use is expanding to the healthcare and beauty industries. Korean Registered Patent No. 10-0539364 relates to a cool massager and operation method using thermoelectric elements.

Conventional healthcare and beauty management methods using thermoelectric devices, including Korean Registered Patent No. 10-0539364, are static massage methods in which the skin is contracted through a constant supply of cold heat, or the muscles are relaxed through a constant supply of warm heat. Static massage can help increase circulation and reduce inflammation.

However, it is only an aid, and static massage does not work directly to release stagnant fluids and wastes from the body. Rather, static massage can cause fluid and waste to clump together in the process of dilating or constricting blood vessels. When fluid is retained in the body, carbon dioxide and waste products of cellular metabolism cannot be expelled, resulting in edema and poor blood supply to tissue cells.

For these reasons, there is an urgent need for a massage method that can solve the problems of static massage. Therefore, the present invention proposes a dynamic massage (or, dynamic thermal therapy, DTT), a control method for flexible thermoelectric elements that can be used for such dynamic massage, and a dynamic thermal therapy device using the same.

### DISCLOSURE

### Technical Problem

An object of the present invention is to provide a method of controlling flexible thermoelectric elements that provide a dynamic massage.

Another object of the present invention is to provide a method for controlling flexible thermoelectric elements applicable to dynamic massage utilizing the effect of flowing cold or warm zones.

Another object of the present invention is to provide a dynamic thermal therapy device applicable to dynamic massage.

Another object of the present invention is to provide a dynamic thermal therapy device applicable to dynamic massage utilizing the effect of flowing cold or warm zones.

### Technical Solution

To address the above challenges, a method of controlling a flexible thermoelectric device to provide dynamic thermal therapy according to the present invention may be characterized in that the flexible thermoelectric device comprises a plurality of thermoelectric modules, each of which corresponds to a plurality of regions arranged sequentially, the plurality of thermoelectric modules selectively perform either exothermic or endothermic operation on the plurality of regions by control of a current, and the method comprises controlling a supply of current to the thermoelectric modules in a first state such that a cold sensation is generated in a first region among the plurality of regions; changing the supply of current to the thermoelectric modules to a second state different from the first state such that the cold sensation is moved to a second region adjacent to the first region; and changing the supply of current to the thermoelectric modules to a third state from the second state, such that the cold sensation is moved to a third region adjacent to the second region, wherein the cold sensation is moved along a direction.

Further, the method of controlling the flexible thermoelectric device to provide dynamic thermal therapy according to the present invention may be characterized in that the plurality of regions are sequentially arranged from the first region to m-th region, where m is an integer, and the supply of current to the thermoelectric modules is changed sequentially from the first state to a n-th state to move the cold sensation along the direction from the first region to the m-th region.

Further, the method of controlling the flexible thermoelectric device to provide dynamic thermal therapy according to the present invention may be characterized in that the method further comprises changing the supply of current from the n-th state to the first state.

Further, the method of controlling the flexible thermoelectric device to provide dynamic thermal therapy according to the present invention may be characterized in that the method further comprises controlling the supply of current to the thermoelectric modules to an initial state such that a warm sensation is generated in the plurality of regions; and changing the supply of current from the n-th state to the initial state.

Further, the method of controlling the flexible thermoelectric device to provide dynamic thermal therapy according to the present invention may be characterized in that the first state is a state of supplying current to the thermoelectric modules such that a warm sensation is generated in a region other than the first region, the second state is a state of supplying current to the thermoelectric modules such that a warm sensation is generated in a region other than the second region, and the third state is a state of supplying current to the thermoelectric modules such that a warm sensation is generated in a region other than the third region.

Further, it may be characterized in that the warm sensation is generated when the cold sensation is reached to a preset temperature.

Further, it may be characterized in that the second region comprises a pair of regions that are left-right symmetrical with respect to the first region, and the third region comprises a pair of regions that are left-right symmetrical with respect to the first region.

Further, it may be characterized in that the first region comprises two non-adjacent regions among said plurality of regions.

Further, a method of controlling a flexible thermoelectric device to provide dynamic thermal therapy according to the present invention may be characterized in that the flexible thermoelectric device comprises a plurality of thermoelectric modules, each of which corresponds to a plurality of regions arranged sequentially, the plurality of thermoelectric modules selectively perform either exothermic or endothermic operation on the plurality of regions by control of a current, and the method comprises controlling a supply of current to the thermoelectric modules in a first state such that a warm sensation is generated in a first region among the plurality of regions; changing the supply of current to the thermoelectric modules to a second state different from the first state such that the warm sensation is moved to a second region adjacent to the first region; and changing the supply of current to the thermoelectric modules to a third state from the second state, such that the warm sensation is moved to a third region adjacent to the second region, wherein the warm sensation is moved along a direction.

Further, a dynamic thermal therapy device of controlling a flexible thermoelectric element to provide dynamic thermal therapy according to the present invention may be characterized in that the dynamic thermal therapy device comprises the flexible thermoelectric element having a plurality of thermoelectric modules, each of which corresponds to a plurality of regions arranged sequentially, wherein the plurality of thermoelectric modules selectively perform either exothermic or endothermic operation on the plurality of regions by control of a current; and a controller configured to control a supply of current to the thermoelectric modules in a first state such that a cold sensation is generated in a first region among the plurality of regions, change the supply of current to the thermoelectric modules to a second state different from the first state such that the cold sensation is moved to a second region adjacent to the first region, change the supply of current to the thermoelectric modules to a third state from the second state, such that the cold sensation is moved to a third region adjacent to the second region, and wherein the controller is configured to move the cold sensation along a direction.

### Advantageous Effects

As discussed above, a method of controlling a flexible thermoelectric device to provide dynamic thermal therapy according to the present invention can compress a user's skin by causing a cold or warm sensation to move along a single direction in a plurality of sequentially arranged regions on the flexible thermoelectric elements. Further, the present invention may compress the user's skin along the direction of movement of the cold or warm sensation, thereby providing a rubbing effect on fluid in the body and causing fluid to drain in the direction of lymph nodes.

Furthermore, the method of controlling the flexible thermoelectric device to provide dynamic thermal therapy according to the present invention can prevent lymphatic backflow and effectively mobilize and drain lymph when the cold or warm sensation departing from the start zone reaches the end zone, and then moves back to the start zone and repeats the same control.

As a result, the method of generating an operational temperature change using flexible thermoelectric elements according to the present invention may provide effects such as: i) increased skin elasticity, ii) increased skin moisture, iii) reduced edema, iv) reduced pigmentation, and v) reduced lymphedema in a user.

### DESCRIPTION OF DRAWINGS

FIG. 1 is a conceptual diagram to illustrate a direction of movement for discharging fluid stagnant in the body out of the body.

FIG. 2 is a conceptual diagram to illustrate a method of controlling flexible thermoelectric elements according to the present invention and a structure of flexible thermoelectric elements of a dynamic thermal therapy device.

FIG. 3 is a conceptual diagram for illustrating an arrangement of thermoelectric modules of flexible thermoelectric elements in a method of controlling the flexible thermoelectric elements according to the present invention and the dynamic thermal therapy device.

FIG. 4 is a block diagram to illustrate a dynamic thermal therapy device according to the present invention.

FIG. 5 is a flow diagram to illustrate a method of controlling flexible thermoelectric elements according to the present invention.

FIG. 6 is a conceptual diagram to illustrate a method of controlling the flexible thermoelectric elements according to the present invention and a process in which, in the dynamic thermal therapy device, coldness or warmth is sequentially moved from a starting region to a final region, and, upon reaching the final region, is moved back to the starting region and the same control is repeated.

FIG. 7 is a conceptual diagram to illustrate a process of controlling the flexible thermoelectric elements according to the present invention and the dynamic thermal therapy device, wherein the coldness or warmth is moved symmetrically from left to right with respect to the starting region, and when the last region is reached, it is moved back to the starting region and the same control is repeated.

FIG. 8 is a conceptual diagram to illustrate the change in operating temperature of the flexible thermoelectric elements during a method of controlling the flexible thermoelectric elements according to the present invention and the dynamic thermal therapy device, wherein the cold sensation is sequentially moved from a start region to a last region, and when the last region is reached, the cold sensation is moved back to the start region and the same control is repeated.

FIG. 9 is a conceptual diagram to illustrate a method of controlling the flexible thermoelectric elements according to the present invention and a process of moving the cold in a gradient in the dynamic thermal therapy device.

FIG. 10 is a conceptual diagram to illustrate the change in operating temperature of the flexible thermoelectric elements during a method of controlling the flexible thermoelectric elements according to the present invention and in the dynamic thermal therapy device, wherein a plurality of cold or warm sensations are sequentially moved from a starting region to a final region, and upon reaching the final region, the flexible thermoelectric element is moved back to the starting region and the same control is repeated.

FIG. 11 is a conceptual diagram to illustrate a method of controlling the flexible thermoelectric elements according to the present invention and a process of moving a plurality of cold spots in a gradient in the dynamic thermal therapy device.

FIG. 12 is a conceptual diagram to illustrate a method of controlling the flexible thermoelectric elements according to the present invention and the control of a thermoelectric module in the dynamic thermal therapy device.

### MODES OF THE INVENTION

Hereinafter, embodiments disclosed herein will be described in detail with reference to the accompanying drawings, wherein identical or similar components, regardless of drawing designation, will be given the same reference numerals and duplicate descriptions will be omitted. The suffixes “module” and “unit” for components used in the following description are assigned or used only for ease of description and are not intended to have a distinct meaning or role by themselves. In addition, in describing the embodiments disclosed herein, if it is determined that a detailed description of the related prior art would obscure the essence of the embodiments disclosed herein, the detailed description is omitted. In addition, the accompanying drawings are intended only to facilitate understanding of the embodiments disclosed herein, and it is to be understood that the technical ideas disclosed herein are not limited by the accompanying drawings and include all modifications, equivalents, or substitutions that are within the scope of the ideas and technology of the present invention.

Terms containing ordinal numbers, such as first, second, and the like, may be used to describe various components, but said components are not limited by said terms. These terms are used only to distinguish one component from another.

When a component is referred to as being “connected” or “related to” to another component, it is to be understood that it may be directly connected or related to that other component, but that there may be other components in between. On the other hand, when a component is said to be “directly connected” or “directly related to” to another component, it should be understood that there are no other components in between.

The singular expression includes the plural unless the context clearly indicates otherwise.

In this application, the terms “include” or “have” are intended to designate the presence of the features, numbers, steps, operations, components, parts, or combinations thereof recited in the specification, and are not intended to preclude the possibility of the presence or addition of one or more other features, numbers, steps, operations, components, parts, or combinations thereof.

Dynamic thermal therapy (DTT), which is a dynamic massage to be provided herein by a method of controlling flexible thermoelectric elements and a dynamic thermal therapy device (1000) of the present invention for providing a dynamic massage, is a massage method for discharging stagnant waste and lymph out of the body.

Lymph is a colorless or yellowish-white fluid that flows through the lymphatic system and is sometimes referred to as impa in Chinese characters. When blood circulates from the arteries through capillaries to the veins, some blood remains between the cells (called interstitial fluid and tissue fluid), and when it collects in lymph capillaries, it is called lymph. This lymph travels deep within the body to places that blood cannot reach, delivering nutrients to various parts of the body, or draining toxins and waste products from cells through lymph nodes.

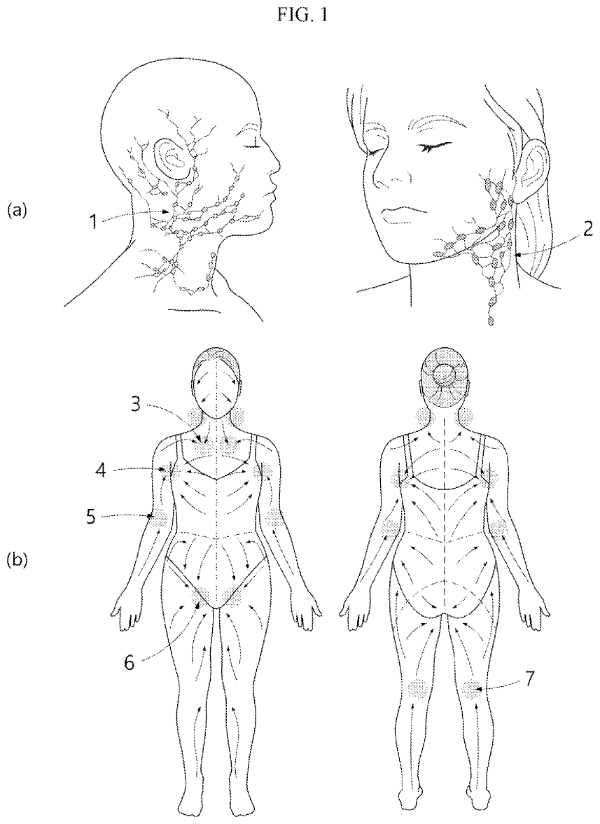

Lymph nodes can be found on the face under the ears (1) and under the chin (2), as shown in FIG. 1(a), or in the collarbone (3), armpits (4), where the arms fold (5), between the thighs (6), and behind the knees (7), as shown in FIG. 1(b).

In order to drain lymph through a lymph node, it is desirable for the lymph to flow in the direction of the lymph node. In the present disclosure, a massage method that utilizes dynamic changes in the thermal environment to induce lymph to flow in the direction of the lymph nodes is referred to as dynamic massage (Dynamic Thermal Therapy, DTT). The dynamic massage of the present invention is not necessarily limited to inducing lymph flow. For example, knees, shoulders, elbows, etc. may be treated through dynamic massage, in which case the dynamic thermal therapy of the present invention may provide dynamic changes in the thermal environment to muscles, nerves, etc. However, for convenience of explanation, dynamic massage will be described hereinafter centering on massage that induces lymph to flow in the direction of lymph nodes. Further, a method for controlling the flexible thermoelectric elements and the dynamic thermal therapy device 1000 for providing dynamic massage according to the present invention will be described below with reference to the following drawings to describe the present invention more specifically.

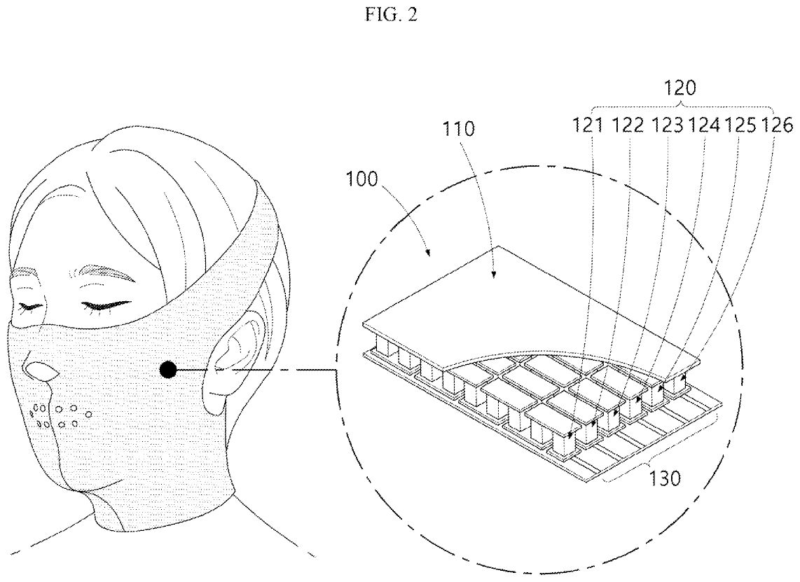

FIG. 2 is a conceptual diagram to illustrate a method for controlling flexible thermoelectric elements according to the present invention and a structure of the flexible thermoelectric element 100 of the dynamic thermal therapy device 1000. The structure of the flexible thermoelectric element 100 according to the present invention will be described with reference to FIG. 2.

The flexible thermoelectric element 100 according to the present invention, which can be freely bent along the bending of the body, can be disposed between a skin contact surface (not shown) that contacts the skin and an outer surface (not shown) that emits heat. Further, the flexible thermoelectric device 100 may include a substrate 110, a plurality of thermoelectric modules 120 disposed between the substrates 110, and a power terminal 130 for applying power to the thermoelectric modules 120.

The substrate 110 serves to support the thermoelectric modules and may be provided with an insulating material. In order for the flexible thermoelectric module 100 to form contact surfaces of different shapes as the body flexes, the substrate 110 may be provided with a flexible material to allow for flexibility. For example, it may be glass fiber or flexible plastic.

The thermoelectric module 120 is disposed on the substrate 110 and may be a pair of different metals (e.g., bismuth and antimony), a pair of N-type and P-type semiconductors, or a plurality thereof electrically connected.

Further, the flexible thermoelectric device 100 according to the present invention may comprise a plurality of thermoelectric modules 120, each corresponding to a plurality of regions arranged sequentially. A “region” may be defined as a range of similar temperatures, formed on a skin contact surface in contact with the skin of a human body, for dynamic thermal therapy. Further, the region may comprise a single thermoelectric module 120 or a plurality of neighboring thermoelectric modules 120. Further, a first region may be used interchangeably with a start region, meaning a region where a temperature change occurs for the first time. The second region may refer to a region where a temperature change occurs at a second time, and the third region may refer to a region where a temperature change occurs at a third time. The m region means the region where the temperature change occurs last, and may be used interchangeably with the last region.

In the flexible thermoelectric element 100 shown in FIG. 2, from the thermoelectric module disposed on the left side to the thermoelectric module disposed on the right side, sequentially referred to as the first thermoelectric module 121, the second thermoelectric module 122, ˜ and the sixth thermoelectric module 126, i) the first region may correspond to the first thermoelectric module 121, the second region may correspond to the second thermoelectric module 122, ˜ and the 6th region may correspond to the sixth thermoelectric module 126. ii) Furthermore, when the first region corresponds to the second thermoelectric module 122, the second region may correspond to the third thermoelectric module 123, the third region may correspond to the fourth thermoelectric module 124, ˜ and the fifth region may correspond to the sixth thermoelectric module 126. iii) Further, the first region may correspond to the first thermoelectric module 121 and the second thermoelectric module 122, the second region may correspond to the third thermoelectric module 123 and the fourth thermoelectric module 124, and the third region may correspond to the fifth thermoelectric module 125 and the sixth thermoelectric module 126. iv) Further, the first region may correspond to the centrally disposed third thermoelectric module 123 and the fourth thermoelectric module 124, the second region may include a pair of regions corresponding to the second thermoelectric module 122 and the fifth thermoelectric module, and the third region may include a pair of regions corresponding to the first thermoelectric module 121 and the sixth thermoelectric module. As such, the “regions” correspond to thermoelectric modules, but may be fluid based on context or user choice.

The thermoelectric module may perform either endothermy or exothermy to the corresponding region by applying a current through the power terminal 130. Said endothermy or exothermy may be determined by the direction of the supplied current, and to this end, the dynamic thermal therapy device 1000 of the present invention is configured to control the direction of the current supplied to the thermoelectric module.

In this case, when the thermoelectric module performs endothermy, a cold sensation may be generated in the corresponding region, and when the thermoelectric module performs exothermy, a warm sensation may be generated in the corresponding region.

Here, the cold sensation may be a temperature that is low enough for the body to feel cold, and may be relatively lower than the temperature when no current is applied to the thermoelectric module. The warm sensation may be a temperature high enough for the body to feel hot, and relatively higher than the temperature when no current is applied to the thermoelectric module.

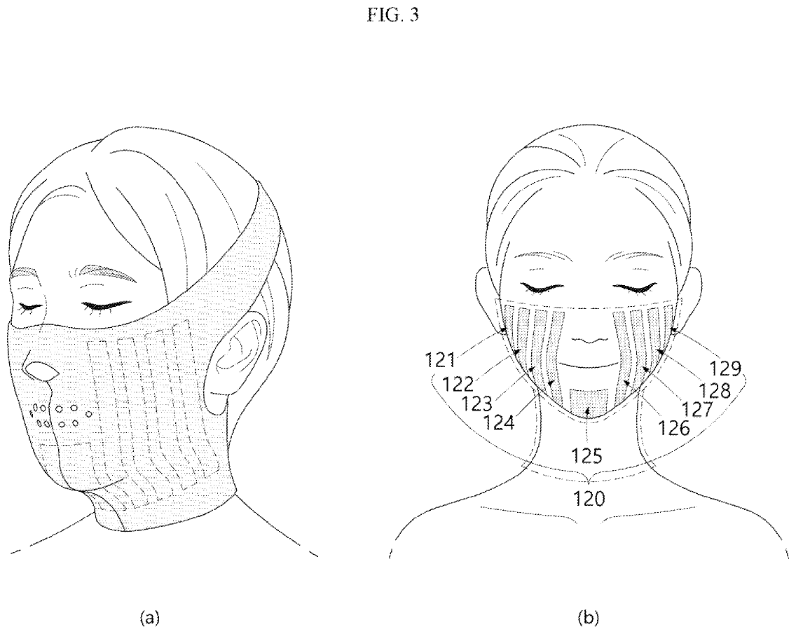

Meanwhile, FIG. 3 is a conceptual diagram to illustrate a method for controlling the flexible thermoelectric elements according to the present invention and an arrangement of thermoelectric modules of the flexible thermoelectric element 100 in the dynamic thermal therapy device 1000. Referring now to FIG. 3, an arrangement of the thermoelectric modules of the flexible thermoelectric element 100 will be described.

Hereinafter, for convenience of explanation, the thermoelectric module disposed on the left side of the flexible thermoelectric element will be referred to as the first thermoelectric module 121, and the thermoelectric modules arranged sequentially from the first thermoelectric module 121 will be referred to as the second thermoelectric module 122, the third thermoelectric module 123, and so on.

The flexible thermoelectric element 100 may be disposed between a skin-contacting surface (not shown) and an outer surface (not shown) of a mask. This is as shown in FIG. 3(a). However, the present invention is not necessarily limited to this, and the flexible thermoelectric element 100 can be applied to various devices for caring for knees, shoulders, elbows, eyes, etc. However, for ease of explanation, the following description will focus on a mask as an example of a dynamic thermal therapy device.

As shown in FIG. 3(b), the plurality of thermoelectric modules may be arranged in sequence (parallel) on the flexible thermoelectric element 100. In this case, the plurality of thermoelectric modules need not be of the same length as each other.

Furthermore, the plurality of thermoelectric modules may be arranged in a left-right symmetry with respect to any one thermoelectric module. In this case, each pair of symmetrical regions may be connected to the same power terminal 130 to receive current.

For example, referring to FIG. 3(b), the first thermoelectric module 121 and the ninth thermoelectric module 129, the second thermoelectric module 122 and the eighth thermoelectric module 128, the third thermoelectric module 123 and the seventh thermoelectric module 127, and the fourth thermoelectric module 124 and the sixth thermoelectric module may be symmetrically disposed relative to the fifth thermoelectric module 125 disposed near the chin of the face. The first thermoelectric module 121 and the ninth thermoelectric module may receive current through the same power terminal 130. As a result, the first thermoelectric module 121 and the ninth thermoelectric module 129 may be controlled to a constant state (either endothermic or exothermic, or a specific temperature) through a single control method.

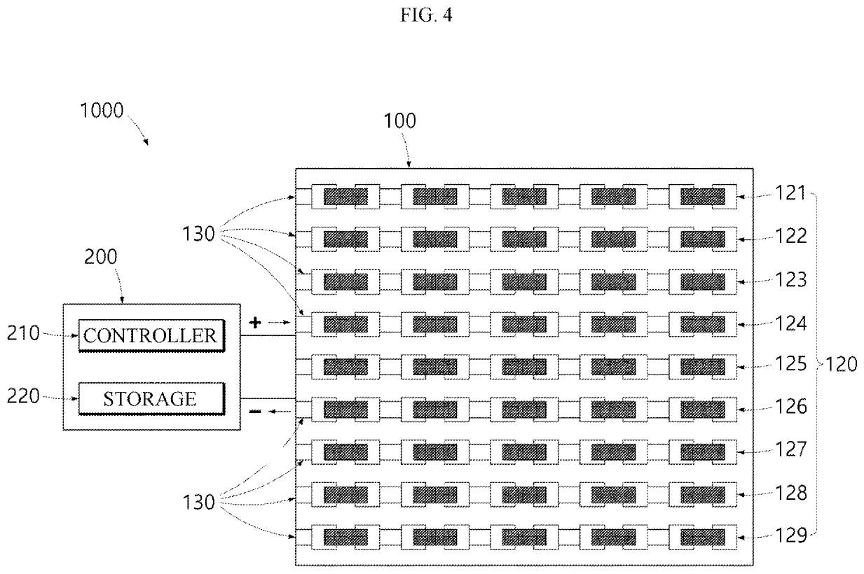

Meanwhile, FIG. 4 is a block diagram to illustrate a dynamic thermal therapy apparatus 1000 according to the present invention. Referring now to FIG. 4, a dynamic thermal therapy device 1000 according to the present invention will be described.

The dynamic thermal therapy device 1000 according to the present invention may include the flexible thermoelectric element 100 described above and a control module 200.

The control module 200 includes a controller 210 and a power source 220, which may further include other configurations.

The power source 220 may supply current to the thermoelectric module via the power terminal 130, under the control of the controller 210, to cause the thermoelectric module to perform endothermic or exothermic operation. Depending on the direction of the current supplied by the power source 220, the thermoelectric module 120 may perform endothermic or exothermic operation. If the thermoelectric module 120 performs endothermy when the power source 220 supplies current in one direction, the thermoelectric module 120 can perform exothermy when the power source 220 supplies current in the other direction opposite to the one direction.

Conversely, if the thermoelectric module 120 performs exothermic operation when the power source 220 supplies current in one direction, the thermoelectric module 120 may perform endothermic operation when the power source 220 supplies current in the other direction opposite to the one direction. Such response of the thermoelectric module 120 according to the direction of the current may be determined based on the structure of the thermoelectric module 120 and user configurations.

However, for ease of explanation, the direction of the current that causes the thermoelectric module 120 to absorb heat will be referred to herein as “forward direction” and the direction of the current that causes the thermoelectric module 120 to generate heat will be referred to as “reverse direction”.

The controller 210 can control the direction of the current supplied by the power source 220 to the thermoelectric modules 120, the intensity of the current, and the duration of the current supply, as well as whether or not to supply current to any of the thermoelectric modules 120. More specifically, the controller 210 may control the supply of current from the power source 220 such that a particular thermoelectric module 120 performs exothermy to reach a certain temperature. Further, the controller 210 may control the current supply of the power source 220 so that the thermoelectric modules 120 arranged side-by-side perform endothermy sequentially, so that the cold sensation generated in the flexible thermoelectric element 100 flows along one direction.

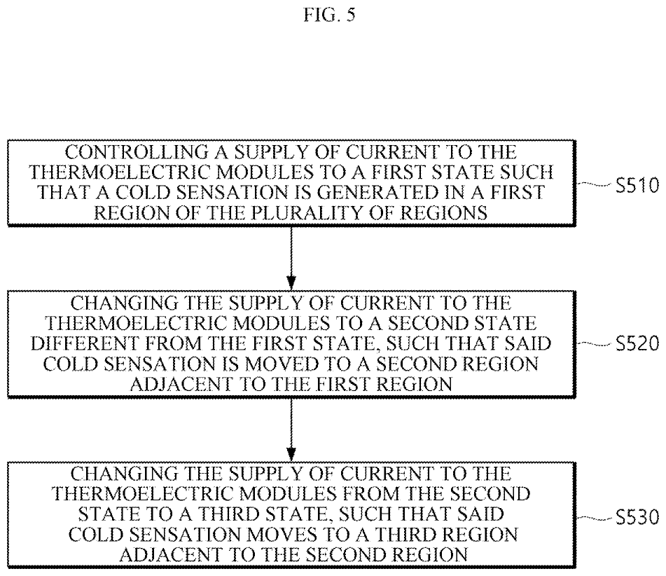

FIG. 5 is a flow diagram to illustrate a method of controlling the flexible thermoelectric elements according to the present disclosure. Referring to FIG. 5, a method of controlling the flexible thermoelectric elements to provide dynamic thermal therapy will be described in more detail.

A method of controlling the flexible thermoelectric element 100 according to the present invention may control a current supply to thermoelectric modules 120 to an initial state such that the warm sensation is generated in a plurality of regions. The initial state may be a state in which the power source 220 is supplying current to the plurality of thermoelectric modules 120 in a reverse direction, i.e., the control unit 210 may control the power source 220 to the initial state so that warmth (or coldness) is generated in the plurality of regions.

In this case, the controller 210 may control the power source 220 such that a constant temperature is generated in the plurality of regions. More specifically, if the plurality of thermoelectric modules 120 have different resistance values, the controller 210 may control the amount of current applied to the plurality of thermoelectric modules 120 differently.

Meanwhile, although the state in which the power source 220 supplies current to the plurality of thermoelectric modules 120 in the reverse direction is named the initial state, a method of controlling the flexible thermoelectric element 100 according to the present invention does not always start with controlling the power source 220 in the initial state. A method of controlling the flexible thermoelectric device 100 according to the present invention may begin with controlling the power source 220 in a first state.

The method of controlling the flexible thermoelectric element according to the present invention may include controlling the current supply to the thermoelectric module 120 to a first state such that a cold sensation is generated in a first region of the plurality of regions (S510).

Here, the first region may be any one of the plurality of regions, as previously described, and may be a region corresponding to a single thermoelectric module 120 or a plurality of neighboring thermoelectric modules 120. More specifically, the first region is the region where the temperature change occurs first, and may be: i) corresponding to a first thermoelectric module 121 disposed at one end of the plurality of thermoelectric modules 120 arranged in sequence. ii) It may also correspond to a thermoelectric module 120 disposed in the middle of the plurality of thermoelectric modules 120 arranged in sequence. iii) Furthermore, the controller 210 may sense the position of the lymph node through the sensor unit (not shown) and identify the area corresponding to the thermoelectric module 120 disposed at the farthest distance from the lymph node as the first area. iv) Further, the controller 210 may receive input from a user of the specific thermoelectric module 120 via the input unit (not shown), and may identify the first region based on the input from the user. v) Further, the first region may include two regions that are not adjacent to each other. For example, the first region may include a region corresponding to a first thermoelectric module 121 and a region corresponding to a third thermoelectric module 123 that is not adjacent thereto.

The first state may be a state in which the power source 220 supplies current in a forward direction to the thermoelectric modules 120 corresponding to the first region. That is, the controller 210 may control the power source 220 to the first state so that a cold sensation is generated in the first region.

Further, the first state may be a state of supplying current in the reverse direction to a thermoelectric module that does not correspond to the first region. That is, the controller 210 may control the power source 220 to the first state so that a cold sensation is generated in the first region and a warm sensation is generated in the other region.

Further, the first state may be a state in which the power source 220 supplies current in a forward direction to the thermoelectric module 120 corresponding to the first region for a predetermined time or until the first region reaches a predetermined lowest temperature (TL). In other words, by controlling the power source 220 to the first state, the controller 210 may control the temperature of the first region to only decrease to a certain temperature (the predetermined lowest temperature, TL).

Meanwhile, the controller 210 may control the power source 220 to a first state for a preset time, thereby supplying a current to the thermoelectric module 120 corresponding to the first region in a forward direction for a preset time. In this case, the thermoelectric module 120 receiving the current may perform heat absorption during the preset time, and the first region may gradually increase the intensity of the cold sensation during the preset time. That is, the first region may gradually decrease in temperature during the preset time.

As a result, the method of controlling the flexible thermoelectric element 100 according to the present invention can deliver a cold sensation to a localized area of the body and, by compressing the area where the cold sensation is delivered, collect lymph (including toxins or wastes) in the body and drain it to a desired location.

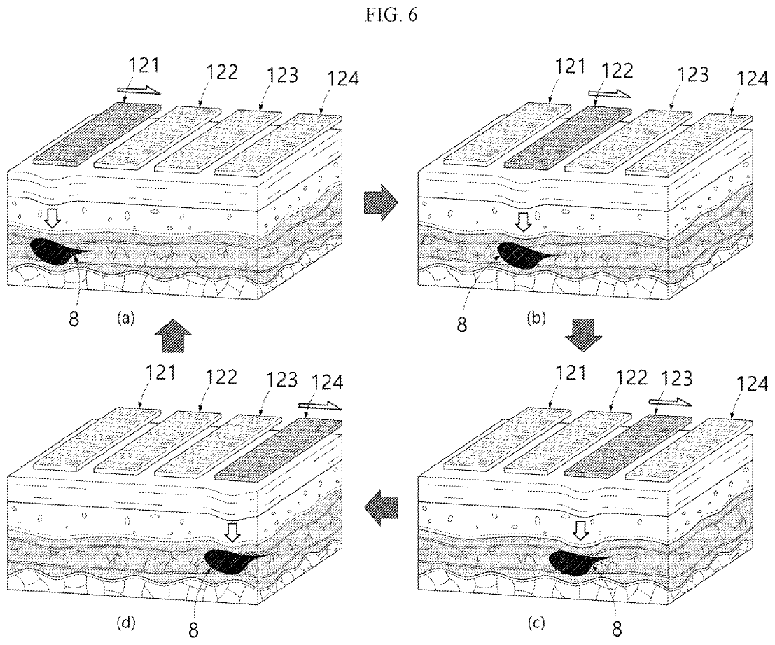

For example, as shown in FIG. 6(a), the controller 210 may control the power source 220 to apply a forward current to the first thermoelectric module 121 corresponding to the first region, and a reverse current to the second through fourth thermoelectric modules 122, 123, 234 corresponding to the other regions. With such control, a cold sensation may be generated in the first region and a warm sensation may be generated in the other regions. Thus, the skin receiving the cold sensation from the first region may compress the lymphatic vessels to move the lymph 8.

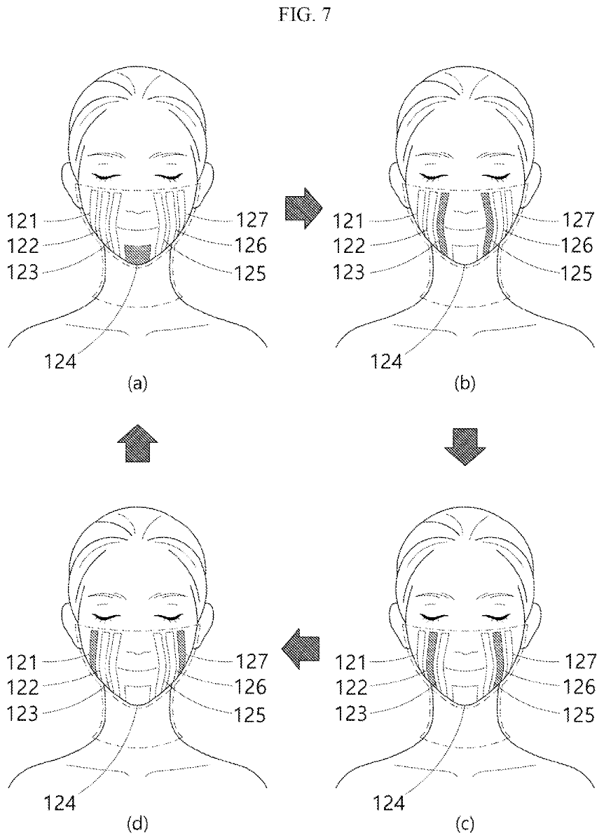

Further, as shown in FIG. 7(a), the controller 210 can control the power supply 220 to apply a forward direction current to the fourth thermoelectric module 124 corresponding to the first region, and a reverse direction current to the first to third and fifth to seventh thermoelectric modules 121, 122, 123, 125, 126, 127 corresponding to the other regions. With such control, a cold sensation may be generated in the first region and a warm sensation may be generated in the other regions. Thus, the skin receiving the cold sensation from the first region may compress the lymphatic vessels to move lymph.

A method of controlling a flexible thermoelectric element according to the present invention can be controlled by changing the current supply to the thermoelectric module 120 to a second state different from the first state, such that the cold sensation is moved to a second region adjacent to the first region (S520).

Here, the second region is a region adjacent to the first region, and may be a region corresponding to a thermoelectric module 120 adjacent to the thermoelectric module 120 corresponding to the first region. More specifically, i) if the first region corresponds to a first thermoelectric module 121, the second region may be a region corresponding to a second thermoelectric module 122. ii) Furthermore, if a total of five regions are arranged sequentially, and the first region located in the middle corresponds to the third thermoelectric module 123, the second region may be a region corresponding to the second thermoelectric module 122 and the fourth thermoelectric module 124.

Meanwhile, the second state may be a state in which the power source 220 supplies current in a forward direction to the thermoelectric module 120 corresponding to the second region of the plurality of thermoelectric modules 120. In other words, the controller 210 may control the power source 220 to the second state so that a cold sensation is generated in the second region. When the controller 210 controls the power source 220 by changing it from the first state to the second state, the cold sensation generated in the flexible thermoelectric element 100 may move in one direction.

Further, the second state may be a state in which the power source 220 supplies current in the reverse direction to the thermoelectric module 120 that does not correspond to the second region. That is, by controlling the power source 220 to the second state, the controller 210 may control that a cold sensation is generated in the second region and a warm sensation is generated in the other region. This can cause a cold sensation with a certain sized area (e.g., a sized area corresponding to a region) to flow along one direction on the flexible thermoelectric element 100.

Further, the second state may be a state in which the power source 220 supplies current in a forward direction to the thermoelectric module 120 corresponding to the second region for a predetermined time or until the second region reaches a predetermined lowest temperature (TL). That is, by controlling the power source 220 to the second state, the controller 210 may control the temperature of the second region to only decrease to a certain temperature (the predetermined lowest temperature, TL).

Further, the second state may be a state in which current is supplied in the reverse direction to the thermoelectric module 120 corresponding to the first region when the second region reaches the preset lowest temperature TL. In such a case, the temperature of the first region may rise relatively slowly compared to the case of supplying a forward current to the thermoelectric module 120 corresponding to the second region while at the same time supplying a reverse current to the thermoelectric module 120 not corresponding to the second region. In other words, the controller 210 may control the power source 220 to the second state to form a temperature gradient from the first region to the second region.

As a result, the method of controlling the flexible thermoelectric element according to the present invention can move the lymph in a direction by moving the localized area of the body where the cold sensation is delivered in the direction. Furthermore, the method of controlling the flexible thermoelectric element according to the present invention can have the same effect as rubbing the lymph in the body in one direction by delivering a gradational cold sensation to a localized area of the body and pressing the area where the gradational cold sensation is delivered with different forces.

For example, as shown in FIG. 6(b), the controller 210 can control the power source 220 to apply a forward direction current to the second thermoelectric module 122 corresponding to the second region, and a reverse direction current to the first, third, and fourth thermoelectric modules 121, 123, 124 corresponding to the other regions. With this control, a cold sensation may be generated in the second region and a warm sensation may be generated in the other regions. Following the first region, the skin receiving the cold sensation from the second region may compress the lymphatic vessels as if rubbed to move the lymph 8 toward the lymph nodes.

Further, as shown in FIG. 7(b), the controller 210 can control the power source 220 such that the power source 220 applies a forward directional current to the third thermoelectric module 123 and the fifth thermoelectric module 125 corresponding to the second region, and a reverse directional current to the first, second, fourth, sixth, and seventh thermoelectric modules 121, 122, 124, 126, and 127 corresponding to the other regions. With this control, a cold sensation may be generated in the second region and a warm sensation may be generated in the other regions. Following the first region, the skin receiving the cold sensation from the second region may compress the lymphatic vessels as rubbed to move lymph toward the lymph nodes (left and right sides of the face).

A method of controlling a flexible thermoelectric element according to the present invention may be controlled by changing a current supply to the thermoelectric module 120 to a third state different from the second state, such that the cold sensation is moved to a third region adjacent to the second region (S520).

Here, the third region may be a region adjacent to the second region, such that the thermoelectric module 120 corresponds to a thermoelectric module adjacent to the second region. Note that the third region may be a region disposed in a position opposite to the first region.

More specifically, i) if the second region corresponds to the second thermoelectric module 122, the third region may be a region corresponding to the third thermoelectric module 123. ii) Furthermore, if a total of five regions are arranged sequentially, and the first region located in the middle corresponds to the third thermoelectric module 123, and the second region corresponds to the second thermoelectric module 122 and the fourth thermoelectric module 124, the third region may be a region corresponding to the first thermoelectric module 121 and the fifth thermoelectric module 125.

Meanwhile, the third state may be a state in which the power source 220 supplies current in a forward direction to the thermoelectric module 120 corresponding to the third region of the plurality of thermoelectric modules. In other words, the controller 210 may control the power source 220 to the third state so that a cold sensation is generated in the third region. When the controller 210 controls the power source 220 by changing the power source 220 from the second state to the third state, the cold sensation generated in the flexible thermoelectric device 100 may move in one direction.

Further, the third state may be a state in which the power source 220 supplies current in the reverse direction to the thermoelectric module 120 that does not correspond to the third region. In other words, by controlling the power source 220 to the third state, the controller 210 may be controlled to generate cold sensation in the third region and warm sensation in the other regions. This can cause a cold sensation with a certain sized area (e.g., a sized area corresponding to a region) to flow along a direction on the flexible thermoelectric element 100.

Further, the third state may be a state in which the power source 220 supplies current in a forward direction to the thermoelectric module 120 corresponding to the third region for a predetermined time or until the third region reaches a predetermined low temperature (TL). In other words, by controlling the power source 220 to the third state, the controller 210 may control the temperature of the third region to only decrease to a certain temperature (the predetermined lowest temperature, TL).

Further, the third state may be a state in which current is supplied to the thermoelectric module 120 corresponding to the second region in the reverse direction when the third region reaches the preset lowest temperature (TL). In such a case, the temperature of the second region may rise relatively slowly compared to the case of supplying a forward direction of current to the thermoelectric module 120 corresponding to the third region while at the same time supplying a reverse direction of current to the thermoelectric module 120 corresponding to the second region. In other words, the controller 210 may control the power supply portion 220 from the first state to the third state in turn, forming a temperature gradient from the first region to the third region.

As a result, the method of controlling the flexible thermoelectric device according to the present invention can move the lymph in one direction by moving the localized area of the body where the cold sensation is delivered in one direction. Furthermore, the method of controlling the flexible thermoelectric element according to the present invention can have the same effect as rubbing the lymph in the body in a unidirectional direction by delivering a gradient cold sensation to a localized area of the body and compressing the area where the gradiated cold sensation is delivered with different forces.

For example, as shown in FIG. 6(c), the controller 210 may control the power source 220 such that the power source 220 applies a forward current to the third thermoelectric module 123 corresponding to the third region, and a reverse current to the first, second, and fourth thermoelectric modules 121, 122, 124 corresponding to the other regions. With this control, a cold sensation may be generated in the third region and a warm sensation may be generated in the other regions. Following the first and second regions, the skin receiving the cold sensation from the third region may compress the lymphatic vessels as if rubbed to move the lymph 8 toward the lymph nodes.

Further, as shown in FIG. 7(c), the controller 210 can control the power source 220 to apply a forward current to the second thermoelectric module 122 and the sixth thermoelectric module 126 corresponding to the third region, and a reverse current to the first, third through fifth, and seventh thermoelectric modules 121, 123, 124, 125, and 127 corresponding to the other regions. With such control, a cold sensation may be generated in the third region and a warm sensation in the other regions. Following the first and second regions, the skin receiving the cold sensation from the third region may compress the lymphatic vessels as if rubbed to move lymph toward the lymph nodes (left and right sides of the face).

As such, a method of controlling flexible thermoelectric elements according to the present invention may control the plurality of regions of the first region to the third region to the first state to the third state, thereby controlling the unidirectional flow of the cold sensation on the flexible thermoelectric element 100. However, in order for the method of controlling the flexible thermoelectric element according to the present invention to be applied to a larger body area (e.g., from the calf to the back of the knee 7 in FIG. 1(b)), or to provide a more thorough rubbing of the body, the plurality of regions may include more regions than three regions. That is, the plurality of regions of the method of controlling the flexible thermoelectric elements according to the present invention may be formed by being sequentially arranged from the first region to the m-th (where m is an integer) region.

Further, the method of controlling the flexible thermoelectric elements according to the present invention can be controlled by sequentially changing the current supply to the thermoelectric module 120 from the first state to the n-th state so as to move along a direction (the direction from the first region to the third region) from the first region to the m-th region.

Here, m is an arbitrary integer to represent the number of the plurality of regions.

When m is 10, the plurality of regions may include a first region, a second region adjacent to the first region, a third region adjacent to the second region, a fourth region adjacent to the third region, through to a tenth region adjacent to the ninth region.

Furthermore, the m-th region may: i) correspond to an opposite side of the first thermoelectric module 121 disposed on one side of the plurality of thermoelectric modules 120 arranged in sequence, that is, to a thermoelectric module 120 disposed on the other side. ii) It may also correspond to a thermoelectric module disposed at a position farthest to the left and right from the thermoelectric module 120 disposed in the middle among the plurality of thermoelectric modules 120 arranged in sequence. iii) Further, the controller 210 may sense the location of the lymph node by means of the sensor unit (not shown), and identify the region corresponding to the thermoelectric module 120 disposed where the lymph node is located as the m-th region. iv) Further, the controller 210 may receive a specific thermoelectric module 120 input from a user via the input unit (not shown), and may designate the m-th region based on the user's input.

Meanwhile, n is a sequentially increasing integer, which may mean from 1 to m. That is, when m is 10, n may be 1, 2, 3 to 9, or 10. Thus, the first region to the m-th region may be represented by the n-th region.

The n-th state (the first state to the m-th state) may refer to a state of forward supplying current to the thermoelectric module 120 corresponding to the n-th region (any of the regions from the first region to the m-th region). More specifically, the state of supplying current in a forward direction to the thermoelectric module 120 corresponding to the first region is referred to as the first state, the state of supplying current in a forward direction to the thermoelectric module 120 corresponding to the second region is referred to as the second state, and the state of supplying current in a forward direction to the thermoelectric module 120 corresponding to the third region is referred to as the third state, the state of supplying current in a forward direction to the thermoelectric module 120 corresponding to the fourth region as the fourth state, the state of supplying current in a forward direction to the thermoelectric module 120 corresponding to the fifth region as the fifth state, through to the state of supplying current in a forward direction to the thermoelectric module 120 corresponding to the tenth region as the tenth state. In other words, the controller 210 can sequentially control the power source 220 to the n-th state (from the first state to the m-th state) so that a cold sensation is generated by moving in one direction from the first region to the m-th region. In this case, the cold sensation generated in the flexible thermoelectric element 100 may move in one direction.

Further, the n-th state (the first state to the m-th state) may refer to a state of supplying current in a reverse direction to the thermoelectric module 120 that does not correspond to the n-th region (any one of the regions from the first region to the m-th region). More specifically, supplying current in the reverse direction to the thermoelectric module 120 not corresponding to the first region is referred to as the first state, supplying current in the reverse direction to the thermoelectric module 120 not corresponding to the second region is referred to as the second state, and supplying current in the reverse direction to the thermoelectric module 120 corresponding to the third region is referred to as the third state, the state of supplying current in the reverse direction to the thermoelectric module 120 not corresponding to the fourth region as the fourth state, the state of supplying current in the reverse direction to the thermoelectric module 120 not corresponding to the fifth region as the fifth state, through to the state of supplying current in the reverse direction to the thermoelectric module 120 not corresponding to the tenth region as the tenth state. In other words, the controller 210 may sequentially control the power source 220 to the n-th state (from the first state to the m-th state) so that a cold sensation having a certain size area (e.g., a size area corresponding to a region) flows along one direction on the flexible thermoelectric element 100, and a warm sensation is generated in other regions.

Further, the n-th state (from the first state to the m-th state) may be a state in which the power source 220 supplies current in a forward direction to the thermoelectric module 120 corresponding to the n-th region for a preset time or until the n-th region (from the first region to the m-th region) reaches a preset lowest temperature (TL). In other words, the controller 210 may control the power source 220 to a third state such that the temperature of the third region is controlled to decrease only to a certain temperature (the preset lowest temperature, TL), may control the power source 220 to a fourth state such that the temperature of the fourth region is controlled to decrease only to a certain temperature (the preset lowest temperature, TL), may control the power source 220 to a fifth state such that the temperature of the fifth region is controlled to decrease only to a certain temperature (the preset lowest temperature, TL), and through to may control the power source 220 to a tenth state such that the temperature of the tenth region is controlled to decrease only to a certain temperature (the preset lowest temperature, TL).

Further, the n-th state (from the first state to the m-th state) may be a state in which the current is supplied to the n−1 th region in the reverse direction when the n-th region (from the first region to the m region) reaches the preset lowest temperature (TL). In other words, the controller 210 may control the power source 220 to a fourth state to supply current in reverse direction to the thermoelectric module corresponding to the third region when the temperature of the fourth region reaches the preset lowest temperature (TL), and may control the power source 220 to a fifth state to supply current in reverse direction to the thermoelectric module corresponding to the fourth region when the temperature of the fifth region reaches the preset lowest temperature (TL), and may control the power source 220 to a sixth state to supply current in the reverse direction to the thermoelectric module corresponding to the fifth region when the temperature of the sixth region reaches a predetermined lowest temperature (TL), and ˜ may control the power source 220 to a tenth state to supply current in the reverse direction to the thermoelectric module corresponding to the ninth region when the temperature of the tenth region reaches a predetermined lowest temperature (TL).

As a result, the method of controlling the flexible thermoelectric elements according to the present invention can move the lymph in one direction by moving the localized area of the body to which the cold sensation is delivered in one direction. Furthermore, a method of controlling the flexible thermoelectric elements according to the present invention can have the same effect as rubbing the lymph in the body in a unidirectional direction by delivering a gradated cold sensation to a localized area of the body and compressing the area where the gradated cold sensation is delivered with different forces.

For example, as shown in FIG. 6(d), the controller 210 may control the power source 220 to apply a forward direction current to the fourth thermoelectric module 124 corresponding to the fourth region, and a reverse direction current to the first to third thermoelectric modules 121, 122, 123. With this control, a cold sensation may be generated in the fourth region and a warm sensation may be generated in the other regions. Following the first through third regions, the skin receiving the cold sensation from the fourth region may compress the lymphatic vessels as if rubbed to move the lymph 8 toward the lymph nodes.

Further, as shown in FIG. 7(d), the control portion 210 may control the power source 220 such that the power source 220 applies a forward direction current to the first thermoelectric module 121 and the seventh thermoelectric module 127 corresponding to the fourth region, and a reverse direction current to the other thermoelectric modules 122 to 126. With this control, a cold sensation may be generated in the fourth region and a warm sensation may be generated in the other regions. Following the first through third regions, the skin receiving the cold sensation from the fourth region may compress the lymphatic vessels as if rubbed to move the lymph toward the lymph nodes (in the direction of the left and right sides of the face).

In this way, the process by which the controller 210 changes the state of the power source 220 to the first state or to the nth state is referred to as a cycle in this specification, i.e., the process by which the controller 210 changes the state of the power source 220 to the first state or to the nth state is referred to as a first cycle if it is performed once, a second cycle if it is performed twice, and a third cycle if it is performed three times.

Furthermore, the method of controlling the flexible thermoelectric elements according to the present invention can change the state of the power source 220 back to the first state when the state of the power source 220 is changed from the first state to the nth state. That is, when the power source 220 performs a one-cycle state change, the controller 210 can change the state of the power source 220 back to the first state.

By controlling the power source 220 to perform one cycle of state change, the controller 210 can rub the lymph in one direction (in particular, the direction to which the lymph nodes are located) by moving the localized area of the body, where the cold sensation is delivered, in one direction. However, it is difficult to drain the lymph out of the body by moving the cold sensation in one direction only once. Therefore, it is necessary to control the power source 220 to perform the state change of the cycle repeatedly.

At this time, the controller 210 shall control the power source 220 to repeat the operation from the first state to the n-th state, but not from the n-th state to the n−1 state, but to return to the first state and repeat the above process. This correlates with the purpose of the present invention to provide dynamic thermal therapy (DTT), because when the cold sensation flows in a direction opposite to the one direction (the direction to which the lymph node is located) (i.e., when the power source 220 changes from the n-th to the n−1-th state), the lymph moved in the direction of the lymph node is moved in the opposite direction to the lymph node.

Therefore, the controller 210 according to the present invention can control the power source 220 to change from the n-th state in which a forward direction current is applied to the n-th region to the first state in which a forward direction current is applied to the first region.

Further, the controller 210 may control the power source 220 such that the power source 220 repeats the cycle of the first state to the n-th state described above. In this case, the controller 210 may control the power source 220 to repeat the cycle a preset number of times. The preset number of times may be set based on information received from a user via the input unit (not shown).

For example, as shown in FIGS. 6(d) and 6(a), the controller 210 may control the power source 220 to apply a forward directional current to the fourth thermoelectric module 124, and then again to apply a forward directional current to the first thermoelectric module 121. Further, the power source 220 can be controlled to apply a reverse current to the other thermoelectric modules. With such control, the skin repeatedly receiving the cold sensation from the first region to the fourth region can repeatedly compress the lymphatic vessels as if rubbed, thereby moving the lymph 8 towards the lymph nodes.

Further, as shown in FIGS. 7(d) and 7(a), the controller 210 can control the power source 220 to apply a forward directional current to the first thermoelectric module 121 and the seventh thermoelectric module 127 corresponding to the fourth region, and then again to apply a forward directional current to the fourth thermoelectric module 124 corresponding to the first region. Further, the power source 220 can be controlled to apply a reverse current to the other thermoelectric modules. With such control, the skin repeatedly receiving the cold sensation from the first region to the fourth region can repeatedly compress the lymphatic vessels as if rubbed, thereby moving the lymph toward the lymph nodes.

On the other hand, in the method of controlling the flexible thermoelectric elements according to the present invention, when the state of the power source 220 is changed from the first state to the n-state, the n-state of the power source 220 can be changed to the initial state. That is, when the power source 220 performs one cycle of state change, the controller 210 can change the state of the power source 220 back to the initial state. Further, the controller 210 may control the state of the power source 220 to change from the initial state to the first state. The controller 210 may then control the power source 220 such that the power source 220 repeats the cycle of the first state to the n-state described above. That is, the controller 210 may control the power source 220 to repeat the cycle including the initial state.

The controller 210 may control the power source 220 such that the power source 220 repeats the cycle of the first state to the n-state described above. In this case, the controller 210 may control the power source 220 to repeat the cycle a preset number of times. The preset number of times may be set based on information received from the user via the input unit (not shown).

At this time, whether the power supply unit 220 performs the cycle including the initial state is under the control of the controller 210, and the controller 210 may determine this based on the user information entered through the input unit (not shown).

So far, a method of controlling the flexible thermoelectric elements according to the present invention has been described with respect to the process of moving the cold sensation along one direction. The flexible thermoelectric element 100 according to the present invention can move not only cold sensation but also warm sensation, which will be described below.

The method of controlling the flexible thermoelectric element according to the present invention to move the warm sensation along one direction is the same as the process of moving the cold sensation described above. However, in order to move the warm sensation, the current direction in each state is reversed. Therefore, it is necessary to redefine the n-th state (i.e., the first state to the n-state). For ease of explanation, the state of moving the warmth will be referred to as the n′ state.

The 1′ state may be a state in which the power source 220 supplies current in the reverse direction to the thermoelectric module 120 corresponding to the first region, i.e., the controller 210 may control the power source 220 to the 1′ state so that warmth is generated in the first region.

Further, the 1′ state may be a state of supplying current in the forward direction to the thermoelectric module 120 not corresponding to the first region. That is, by controlling the power source 220 to the 1′ state, the controller 210 may control that a warmth is generated in the first region and a cold sensation is generated in the other regions.

Further, the 1′ state may be a state in which the power source 220 supplies current in reverse to the thermoelectric module 120 corresponding to the first region for a preset time or until the first region reaches a preset highest temperature. In other words, by controlling the power source 220 to the 1′ state, the controller 210 may control the temperature of the first region to rise only to a certain temperature (the preset highest temperature, TH).

On the other hand, by controlling the power source 220 to the first state for a preset time, the controller 210 can supply a current in the reverse direction for a preset time to the thermoelectric module 120 corresponding to the first region. In this case, the thermoelectric module receiving the current performs heating for the preset time, and the first region can gradually increase the intensity of the warmth for the preset time, i.e., the first region can gradually increase the temperature for the preset time.

On the other hand, the 2′ state may be a state in which the power source 220 supplies current in the reverse direction to the thermoelectric module 120 corresponding to the second region of the plurality of thermoelectric modules. That is, the controller 210 may control the power source 220 to the 2′ state so that warmth is generated in the second region. When the controller 210 controls the power source 220 by changing the power source 220 from the first state to the second state, the warmth generated in the flexible thermoelectric device 100 may move in one direction.

Further, the 2′ state may be a state in which the power source 220 supplies current in a forward direction to the thermoelectric module 120 that does not correspond to the second region. In other words, by controlling the power source 220 to the 2′ state, the controller 210 may control that a warmth is generated in the second region and a cold sensation is generated in the other region. As a result, a warm sensation with a certain sized area (e.g., a sized area corresponding to a region) can be caused to flow along a unidirectional path across the flexible thermoelectric element.

Further, the 2′ state may be a state in which the power source 220 supplies current in the reverse direction to the thermoelectric module 120 corresponding to the second region for a preset time or until the second region reaches a preset highest temperature. That is, by controlling the power source 220 to the 2′ state, the control unit 210 may control the temperature of the second region to increase only to a certain temperature (the preset highest temperature, TH).

Furthermore, the 2′ state may be a state in which current is supplied in a forward direction to the first region when the second region reaches the preset highest temperature (TH). In such a case, the temperature of the first region may increase relatively slowly compared to the case of supplying a reverse current to the thermoelectric module 120 corresponding to the second region while at the same time supplying a forward current to the thermoelectric module 120 not corresponding to the second region. In other words, the controller 210 may control the power source 220 to the 2′ state to form a temperature gradation from the first region to the second region.

On the other hand, the 3′ state may be a state in which the power source 220 supplies current in the reverse direction to the thermoelectric module 120 corresponding to a third region of the plurality of thermoelectric modules. That is, the controller 210 may control the power source 220 to the 3′ state so that warmth is generated in the third region. When the controller 210 controls the power source 220 by changing the power source 220 from the second state to the third state, the warmth generated in the flexible thermoelectric element 100 may move in one direction.

Further, the 3′ state may be a state in which the power source 220 supplies current in a forward direction to the thermoelectric module 120 that does not correspond to the third region. In other words, by controlling the power source 220 to the 3′ state, the control unit 210 may control the power source 220 to generate warmth in a third region and cold sensation in another region. This can cause warm sensation with a specific sized area (e.g., a sized area corresponding to a region) to flow unidirectionally across the flexible thermoelectric element 100.

Further, the 3′ state may be a state in which the power source 220 supplies current in the reverse direction to the thermoelectric module 120 corresponding to the third region for a preset time or until the third region reaches a preset highest temperature (TH). That is, by controlling the power source 220 to the 3′ state, the controller 210 may control the temperature of the third region to only decrease to a certain temperature (the preset highest temperature, TH).

Further, the 3′ state may be a state in which current is supplied to the second region in the forward direction when the third region reaches the preset highest temperature. In such a case, the temperature of the second region may decrease relatively slowly compared to the case of supplying a reverse current to the thermoelectric module 120 corresponding to the third region while at the same time supplying a forward current to the thermoelectric module 120 corresponding to the second region. In other words, the controller 210 may control the power source 220 in turn from the 1′ state to the 3′ state to form a temperature gradation from the first region to the third region.

On the other hand, the n′ state (from the 1′ state to the m′ state) may refer to a state of supplying current in the reverse direction to the thermoelectric module 120 corresponding to the n-th region (any of the regions from the first region to the m-th region). More specifically, it can be understood that the state of supplying current in the reverse direction to the thermoelectric module 120 corresponding to the first region is referred to as the 1′ state, the state of supplying current in the reverse direction to the thermoelectric module 120 corresponding to the second region is referred to as the 2′ state, and the state of supplying current in the reverse direction to the thermoelectric module 120 corresponding to the third region is referred to as the 3′ state, supplying current in the reverse direction to the thermoelectric module 120 corresponding to the fourth region as the 4′ state, supplying current in the reverse direction to the thermoelectric module 120 corresponding to the fifth region as the 5′ state, and supplying current in the reverse direction to the thermoelectric module 120 corresponding to the tenth region as the 10′ state. In other words, the controller 210 can sequentially control the power supply 220 to the n′ state (from the 1′ state to the m′ state) so that the warmth is generated by moving in one direction from the first region to the m-th region. In this case, the warmth generated by the flexible thermoelectric element 100 can move in a unidirectional direction.

Further, the n′ state (the 1′ state to the m′ state) may refer to a state of supplying a current in a forward direction to the thermoelectric module 120 that does not correspond to the n-th region (any of the regions from the first region to the m-th region). More specifically, it can be understood that a state of supplying current in a forward direction to the thermoelectric module 120 not corresponding to the first region is referred to as a state of 1′, a state of supplying current in a forward direction to the thermoelectric module 120 not corresponding to the second region is referred to as a state of 2′, and a state of supplying current in a forward direction to the thermoelectric module 120 corresponding to the third region is referred to as a state of 3′, a state of supplying current in a forward direction to the thermoelectric module 120 not corresponding to the fourth region as the 4′ state, a state of supplying current in a forward direction to the thermoelectric module 120 not corresponding to the fifth region as the 5′ state, and a state of supplying current in a forward direction to the thermoelectric module 120 not corresponding to the tenth region as the 10′ state. In other words, the controller 210 may sequentially control the power source 220 to the n′ state (from the 1′ state to the m′ state) such that a warm sensation having a certain sized area (e.g., a sized area corresponding to a region) flows along one direction on the flexible thermoelectric element 100, and cold sensation is generated in other regions.

Further, the n′ state (the 1′ state to the m′ state) may be a state in which the power source 220 supplies current in the reverse direction to the thermoelectric module corresponding to the n-th region for a preset time or until the n-th region (the first region to the m-th region) reaches a preset highest temperature (TH). In other words, the controller 210 may control the power source 220 to the 3′ state to control the temperature of the third region to increase only to a certain temperature (the preset highest temperature, TH), and may control the power source 220 to the 4′ state to control the temperature of the fourth region to increase only to a certain temperature (the preset highest temperature, TH), by controlling the power source 220 to the 5′ state, the temperature of the fifth region can be controlled to increase only up to a certain temperature (a preset highest temperature), and ˜ by controlling the power source 220 to the 10′ state, the temperature of the tenth region can be controlled to increase only up to a certain temperature (a preset highest temperature, TH).

Further, the n′ state (the 1′ state to the m′ state) may be a state in which a current is supplied in a forward direction to the thermoelectric module 120 corresponding to the n−1-th region when the n-th region (the first region to the m-th region) reaches the preset highest temperature (TH). That is, the controller 210 may control the power source 220 to the 4′ state to supply current in a forward direction to the thermoelectric module 120 corresponding to the third region when the temperature of the fourth region reaches the preset highest temperature (TH), and may control the power source 220 to the 5′ state to supply current in a forward direction to the thermoelectric module 120 corresponding to the fourth region when the temperature of the fifth region reaches the preset highest temperature (TH), By controlling the power source 220 to the 6′ state, a current can be supplied in a forward direction to the thermoelectric module corresponding to the fifth region when the temperature in the fifth region reaches a predetermined low temperature (TL), and ˜ By controlling the power source 220 to the 10′ state, a current can be supplied in a forward direction to the thermoelectric module corresponding to the ninth region when the temperature in the tenth region reaches a predetermined high temperature (TH).

As a result, the method of controlling the flexible thermoelectric elements according to the present invention can move the lymph in one direction by moving the localized area of the body to which the warmth is delivered in one direction. Furthermore, the method of controlling the flexible thermoelectric device according to the present invention can have the same effect as rubbing the lymph in the body in a unidirectional direction by delivering a gradational warmth to a localized area of the body and compressing the area where the gradational warmth is delivered with different forces.

So far, a method for controlling the flexible thermoelectric elements according to the present invention has been described in accordance with the movement of the cold or warm sensation. Hereinafter, a method of controlling a flexible thermoelectric element according to the temperature change of a cold sensation will be described.

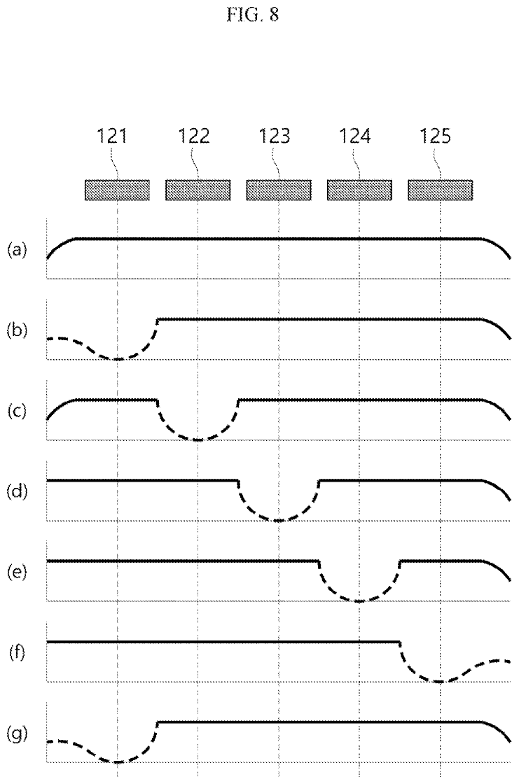

FIG. 8 is a conceptual diagram for illustrating a method for controlling a flexible thermoelectric element according to the present invention and a change in the operating temperature of the flexible thermoelectric element 100 while the cold or warm sensation is sequentially moved from a starting region (first region) to a final region (fifth region) in the dynamic thermal therapy device 1000, and when the final region (fifth region) is reached, it is returned to the starting region (first region) and the same control is repeated. The horizontal axis of each graph illustrated in FIG. 8 may represent a distance s, and the vertical axis may represent a temperature T. Meanwhile, the thermoelectric modules disposed at the top of the graph may refer to the first thermoelectric module 121, the second thermoelectric module 122, to the fifth thermoelectric module 125, from the left.

The method of controlling the flexible thermoelectric element according to the present invention may supply a reverse current to the thermoelectric module 120 corresponding to the first region 121 to the last region 125 such that the thermoelectric module corresponding to the first region 121 to the last region 125 performs exothermal operation. That is, the controller 210 may control the power source 220 such that the power source 220 operates in an initial state (a state in which the reverse current is applied to all thermoelectric modules). As a result, the plurality of zones may reach a preset highest temperature (TH), as illustrated in FIG. 8(a).

A method of controlling a flexible thermoelectric element according to the present invention may apply a current in a forward direction to a first thermoelectric module 121 corresponding to a first region, and a current in a reverse direction to thermoelectric modules 122, 123, 124, 125 corresponding to other regions so that the thermoelectric modules 122, 123, 124, 125 corresponding to other regions perform exothermy. In other words, the controller 210 may control the power source 220 such that the power source 220 operates in a first state (a state in which the power source 220 applies a current in the forward direction only to the first thermoelectric module 121 corresponding to the first region). Further, the controller 210 may stop supplying current to the first thermoelectric module 121 corresponding to the first region when the first region reaches a predetermined lowest temperature (TL). As a result, the first region may generate a cold sensation and the other region may generate a warm sensation, as shown in FIG. 8(b). Further, the first region may gradually decrease in temperature and then gradually increase in temperature upon reaching a preset lowest temperature (TL).

A method of controlling a flexible thermoelectric element according to the present invention may apply a current in a forward direction to the second thermoelectric module 122 corresponding to the second region to perform endothermy, and a current in a reverse direction to the thermoelectric modules 121, 123, 124, 125 corresponding to the other regions to perform exothermy. In other words, the controller 210 may control the power source 220 such that the power source 220 operates in a second state (i.e., a state in which the power source 220 applies forward current only to the thermoelectric modules corresponding to the second region). Further, the controller 210 may stop supplying current to the second thermoelectric module 122 corresponding to the second region when the second region reaches a preset lowest temperature (TL). As a result, the second region may generate a cold sensation and the other region may generate a warm sensation, as shown in FIG. 8(c). Further, the second zone may gradually decrease in temperature and then gradually increase in temperature upon reaching a preset lowest temperature TL.

Similarly, the controller 210 may control the power source 220 such that the power source 220 operates sequentially in a third state (a state in which a forward current is applied only to the third thermoelectric module 123 corresponding to the third region), a fourth state (a state in which a forward current is applied only to the fourth thermoelectric module 124 corresponding to the fourth region), and a fifth state (a state in which a forward current is applied only to the fifth thermoelectric module 125 corresponding to the fifth region). This is illustrated in FIGS. 8(d) to 8(f), respectively.

When the fifth region corresponds to the last region, the controller 210 may perform a first cycle by controlling the power source 220 to operate in the fifth state.

After performing the first cycle, the method of controlling the flexible thermoelectric element according to the present invention may again apply a current in the forward direction to the first thermoelectric module 121 corresponding to the first region to perform heat absorption to the thermoelectric module corresponding to the first region, and a current in the reverse direction to the thermoelectric modules 121, 122, 124, 125 corresponding to the other regions to perform heat generation to the thermoelectric modules 121, 122, 124, 125 corresponding to the other regions. In other words, the controller 210 can control the power source 220 such that the power source 220 operates in the first state again (i.e., a state in which the power source 220 applies a current in the forward direction only to the first thermoelectric module 121 corresponding to the first region). This is illustrated in FIG. 8(g). The method of controlling the flexible thermoelectric element according to the present invention may be controlled to perform a second cycle, starting with FIG. 8(g).

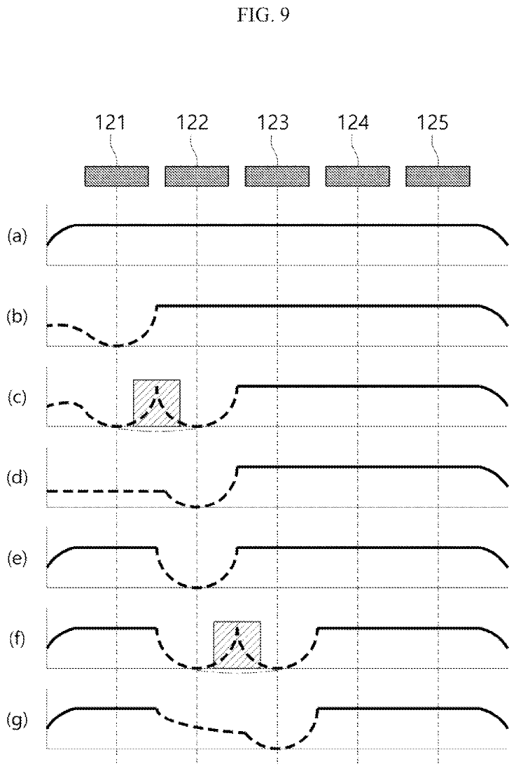

FIG. 9 is a conceptual diagram to illustrate a process in which the cold sensation moves while forming gradations in a method of controlling a flexible thermoelectric element and a dynamic thermal therapy device 1000 according to the present invention. The horizontal axis of each graph illustrated in FIG. 8 may represent a distance s, and the vertical axis may represent a temperature T. Meanwhile, the thermoelectric modules disposed at the top of the graph may refer to, from the left, the first thermoelectric module 121, the second thermoelectric module 122, to the fifth thermoelectric module 125.

The control process for the power source 220 to operate in the initial state (FIG. 9 (a)) and the first state (FIG. 9 (b)), and the change in temperature thereof, are the same as described above.

On the other hand, the controller 210 may control the power source 220 such that the power source 220 supplies current in the reverse direction to the first thermoelectric module 121 corresponding to the first region when the second region reaches a preset lowest temperature (TL). With this control, the temperature (cold or warm) in the first region and the cold sensation in the second region may overlap to form a temperature (i.e., a temperature gradation) where the boundary is similar to the predetermined lowest temperature. In other words, as shown in FIG. 9(c), the overlap of different temperatures in the box portion may form a temperature gradation, as shown in FIG. 9(d), and then concentrate the cold sensation in the second region, as shown in FIG. 9(d).

The controller 210 may control the power source 220 such that the power source 220 supplies current in the reverse direction to the second thermoelectric module 122 corresponding to the second region when the third region reaches a predetermined lowest temperature (TL). With this control, the temperature (cold or warm) of the second region and the cold sensation in the third region may overlap to form a temperature (i.e., a temperature gradation) where the boundary is similar to the preset lowest temperature (TL). That is, when the different temperatures of the box portions overlap, as shown in FIG. 9(f), a temperature gradation may be formed, as shown in FIG. 9(g).

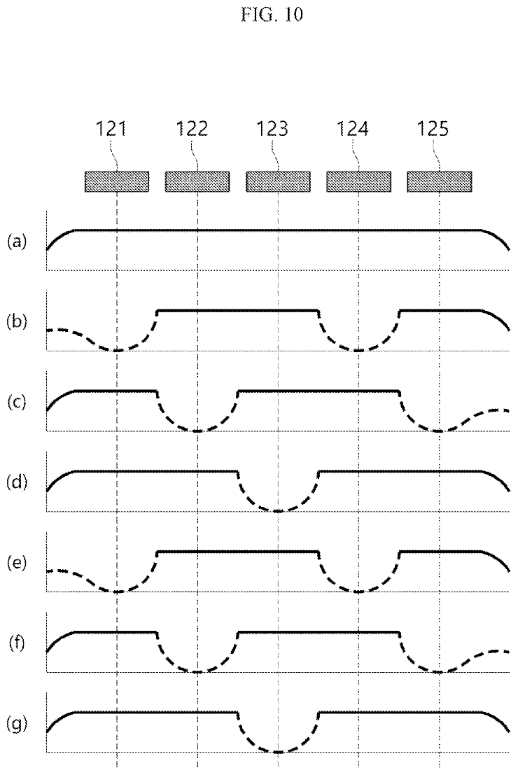

FIG. 10 is a conceptual diagram for illustrating the change in operating temperature of the flexible thermoelectric element 100 during a method of controlling the flexible thermoelectric element according to the present invention and in a dynamic thermal therapy device 1000, wherein a plurality of cold or warm sensations are sequentially moved from a start region to a last region, and upon reaching the last region, the flexible thermoelectric element 100 is returned to the start region and the same control is repeated. The horizontal axis of each graph illustrated in FIG. 8 may represent a distance s, and the vertical axis may represent a temperature T. Meanwhile, the thermoelectric modules disposed at the top of the graph may refer to, from left to right, a first thermoelectric module 121, a second thermoelectric module 122, to a fifth thermoelectric module 125.

A method of controlling a flexible thermoelectric element according to the present invention may apply a current in a forward direction to a thermoelectric module corresponding to a first region such that a thermoelectric module corresponding to the first region performs endothermy, and a current in a reverse direction to a thermoelectric module corresponding to another region such that a thermoelectric module corresponding to the other region performs exothermy. In this case, the first region may include different non-adjacent regions. For example, as shown in FIG. 10(b), the first region may be a region corresponding to the first thermoelectric module 121 and a region corresponding to the fourth thermoelectric module 124.

In other words, the controller 210 may control the power source 220 such that the power source 220 operates in a first state (a state in which a forward current is applied only to the first thermoelectric module 121 and the fourth thermoelectric module 124 corresponding to the two first regions). Further, the controller 210 may stop supplying current to the first thermoelectric module 121 and the fourth thermoelectric module 124 corresponding to the two first regions when the two first regions reach a predetermined lowest temperature (TL). As a result, the two first regions may generate a cold sensation and the other regions may generate a warm sensation, as illustrated in FIG. 10(b). Further, the two first regions may gradually decrease in temperature and then gradually increase in temperature when a preset lowest temperature (TL) is reached.

A method of controlling a flexible thermoelectric element according to the present invention may apply a current in a forward direction to a thermoelectric module corresponding to a second region to perform endothermy, and a current in a reverse direction to a thermoelectric module corresponding to another region to perform exothermy. In this case, the second region may include a region adjacent to each of the different first regions. For example, as shown in FIG. 10(c), the second region may include a region corresponding to the second thermoelectric module 122 and a region corresponding to the fifth thermoelectric module 125.

In other words, the controller 210 may control the power source 220 such that the power source 220 operates in a second state (a state in which the power source 220 applies a forward current only to the second thermoelectric module 122 and the fifth thermoelectric module 125 corresponding to the second region). Further, the controller 210 may stop supplying current to the second thermoelectric module 122 and the fifth thermoelectric module 125 corresponding to the two second regions when the two second regions reach a predetermined lowest temperature (TL). This may cause the two second regions to generate a cold sensation and the other regions to generate a warm sensation, as illustrated in FIG. 10(c). Furthermore, the two second regions may gradually decrease in temperature and gradually increase in temperature upon reaching a predetermined lowest temperature (TL).

A method of controlling a flexible thermoelectric element according to the present invention may apply a current in a forward direction to a thermoelectric module corresponding to a third region to perform endothermy, and a current in a reverse direction to a thermoelectric module corresponding to another region to perform exothermy. In this case, the third region may comprise a region adjacent to each of the different second regions. For example, as shown in FIG. 10(d), the third region may include a region corresponding to a third thermoelectric module 123.

However, since the embodiment of FIG. 10(d) includes a fifth thermoelectric module 125, the third region may include only one region corresponding to the third thermoelectric module 123.

In other words, the controller 210 may control the power source 220 such that the power source 220 operates in a third state (i.e., a state in which a forward current is applied only to the thermoelectric modules corresponding to the third region). Further, the controller 210 may stop supplying current to the third thermoelectric module 123 corresponding to the third region when the third region reaches a predetermined lowest temperature. As a result, the third region may generate a cold sensation and the other regions may generate a warm sensation, as illustrated in FIG. 10(d). Further, the third region may have a gradual decrease in temperature and a gradual increase in temperature upon reaching a preset lowest temperature (TL).

Meanwhile, the controller 210 may determine that the two zones can no longer be identified as a pair, that is, if only one zone has been identified, the controller 210 may identify that zone as the last zone and end the first cycle. Thereafter, the controller 210 may control the power source 220 to operate the power source 220 in the first state to the third state again, as shown in FIGS. 10(e) to 10(g).

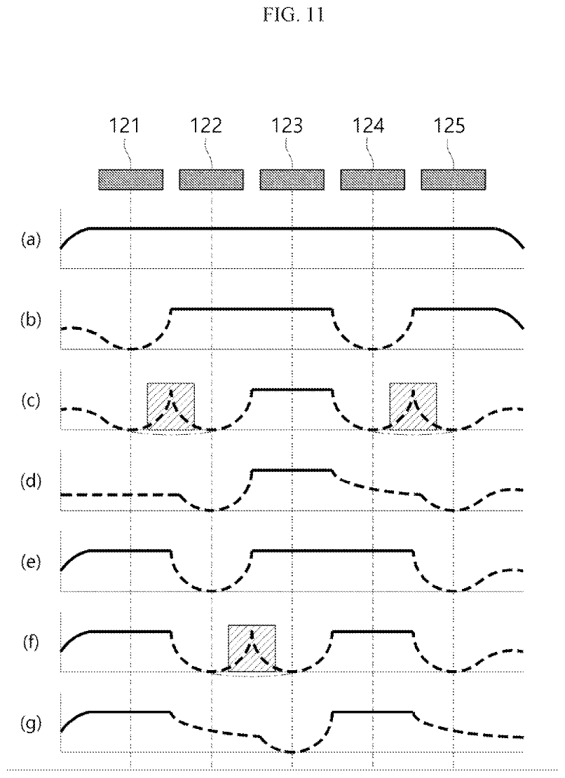

FIG. 11 is a conceptual diagram to illustrate a process in which the plurality of cold sensations move in forming a gradation in a method of controlling a flexible thermoelectric element and a dynamic thermal therapy device 1000 according to the present invention. The horizontal axis of each graph illustrated in FIG. 11 may represent a distance s, and the vertical axis may represent a temperature T. Meanwhile, the thermoelectric module 120 disposed at the top of the graph may refer to, from the left, the first thermoelectric module 121, the second thermoelectric module 122, to the fifth thermoelectric module 125.

The control process for the power source 220 to operate in the initial state (FIG. 11, (a)) and the first state (FIG. 11, (b)), and the change in temperature accordingly, are the same as described above.

On the other hand, the controller 210 may control the power source 220 such that the power source 220 supplies current in the reverse direction to the first thermoelectric module 121 corresponding to the first region when the second region reaches a preset lowest temperature (TL). With this control, the temperature (cold or warm) of the first region and the coldness of the second region may be superimposed to form a temperature (i.e., a temperature gradation) where the boundary is similar to the predetermined lowest temperature (TL). In other words, as shown in FIG. 11(c), the superimposition of different temperatures of the box portion may form a temperature gradient, as shown in FIG. 11(d), and then concentrate the coldness in the second region, as shown in FIG. 11(d).

The controller 210 may control the power source 220 such that the power source 220 supplies current in the reverse direction to the third thermoelectric module 123 corresponding to the third region when the third region reaches a predetermined lowest temperature (TL). With this control, the temperature (cold or warm) of the second region and the cold sensation of the third region may be superimposed to form a temperature (i.e., a temperature gradation) where the boundary is similar to the preset lowest temperature (TL). That is, when the different temperatures of the box portions are superimposed, as shown in FIG. 11(f), a temperature gradation may be formed, as shown in FIG. 11(g).

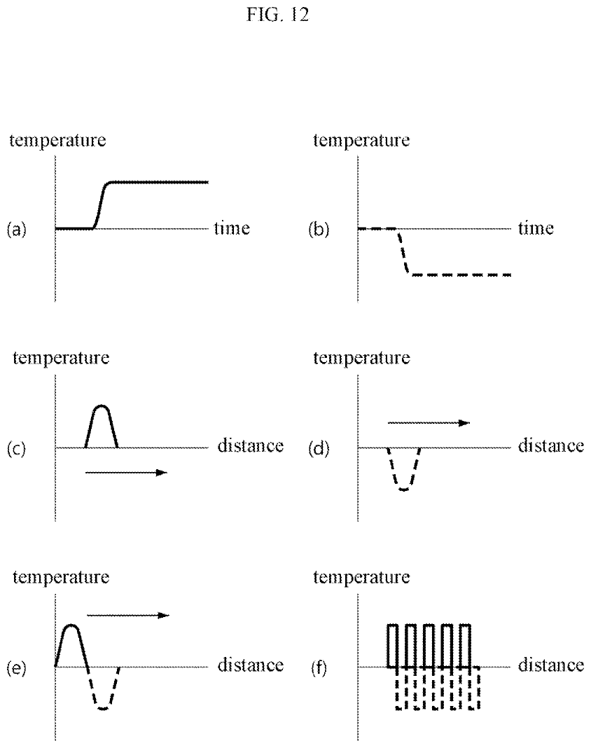

FIG. 12 is a conceptual diagram to illustrate a control of a thermoelectric module in a method of controlling the flexible thermoelectric elements and the dynamic thermal therapy device 1000 according to the present invention.

The controller 210 can control i) the temperature, ii) the rate of temperature increase (i.e., the amount of temperature change), and iii) the duration of the temperature, of the flexible thermoelectric element 100. More specifically, the controller 210 may control the i) temperature, ii) rate of temperature increase (i.e., amount of temperature change), and iii) temperature duration differently for a plurality of thermoelectric modules. This is illustrated in FIGS. 12(a) and 12(b).

Further, the controller 210 can control the i) temperature, ii) rate of temperature increase (i.e., amount of temperature change), and iii) temperature duration for a plurality of neighboring thermoelectric modules 120. As a result, the controller 210 can control the i) temperature, ii) rate of temperature increase (i.e., amount of temperature change), and iii) temperature duration in units of a particular sized area of the flexible thermoelectric element 100 (e.g., a sized area corresponding to a particular region). This is illustrated in FIGS. 12(c) and 12(d).

Further, the controller 210 can control the flexible thermoelectric device 100 to move in an alternating manner between hot and cold. More specifically, the controller 210 may cause the power source 220 to apply a current in a reverse direction to the first thermoelectric module 121 to generate a warm sensation in a first region, and to apply a current in a forward direction to the second thermoelectric module 122 to generate a cold sensation in a second region. The controller 210 may control the power source 220 to power the plurality of thermoelectric modules such that the warmth and coolness flow along a unidirectional path. For example, a sequentially reverse current may be applied to the odd numbered regions (first, third, fifth, seventh, and ninth regions) to generate warm sensation, and a sequentially forward current may be applied to the even numbered regions (second, fourth, sixth, eighth, and tenth regions) to generate cold sensation. This is as illustrated in FIGS. 12(e) and 12(f). In other words, the controller 210 can simultaneously deliver cold and warm sensations to a portion of the skin, allowing the user to feel a sense of pain.

As shown above, a method of controlling a flexible thermoelectric element to provide dynamic thermal therapy in accordance with the present invention can compress a user's skin by causing cold or warmth to travel along a unidirectional path in a plurality of sequentially arranged regions on the flexible thermoelectric element 100. Further, by compressing the user's skin in accordance with the direction of movement of the cold or warm sensation, the present invention can provide a rubbing effect on fluid in the body and cause fluid to move and drain toward the lymph nodes (forward).

Furthermore, the method of controlling the flexible thermoelectric device to provide dynamic thermal therapy according to the present invention can prevent lymphatic reflux and effectively mobilize lymphatic drainage by providing the same control repeatedly after the cold or warm sensation reaches the end zone and returns to the start zone.

As a result, the method of generating an operational temperature change using the flexible thermoelectric element 100 according to the present invention can provide effects such as i) increasing skin elasticity, ii) increasing skin moisture, iii) reducing edema, iv) reducing pigmentation, and v) reducing lymphedema in breast cancer patients.

1. A method for controlling a flexible thermoelectric device to provide dynamic thermal therapy, wherein the flexible thermoelectric device comprises a plurality of thermoelectric modules, each of which corresponds to a plurality of regions arranged sequentially, and wherein the plurality of thermoelectric modules selectively perform either exothermic or endothermic operation on the plurality of regions by control of a current, the method comprising:
controlling a supply of current to the thermoelectric modules in a first state such that a cold sensation is generated in a first region among the plurality of regions;
changing the supply of current to the thermoelectric modules to a second state different from the first state such that the cold sensation is moved to a second region adjacent to the first region; and
changing the supply of current to the thermoelectric modules to a third state from the second state, such that the cold sensation is moved to a third region adjacent to the second region,
wherein the cold sensation is moved along a direction.

2. The method of claim 1,
wherein the plurality of regions are sequentially arranged from the first region to m-th region, where m is an integer, and
wherein the supply of current to the thermoelectric modules is changed sequentially from the first state to a n-th state to move the cold sensation along the direction from the first region to the m-th region.

3. The method of claim 2, further comprising:
changing the supply of current from the n-th state to the first state.

4. The method of claim 2, further comprising:
controlling the supply of current to the thermoelectric modules to an initial state such that a warm sensation is generated in the plurality of regions; and
changing the supply of current from the n-th state to the initial state.

5. The method of claim 1,
wherein the first state is a state of supplying current to the thermoelectric modules such that a warm sensation is generated in a region other than the first region,
wherein the second state is a state of supplying current to the thermoelectric modules such that a warm sensation is generated in a region other than the second region, and
wherein the third state is a state of supplying current to the thermoelectric modules such that a warm sensation is generated in a region other than the third region.

6. The method of claim 5,
wherein the warm sensation is generated when the cold sensation is reached to a preset temperature.

7. The method of claim 1,
wherein the second region comprises a pair of regions that are left-right symmetrical with respect to the first region, and
wherein the third region comprises a pair of regions that are left-right symmetrical with respect to the first region.

8. The method of claim 1,
wherein the first region comprises two non-adjacent regions among said plurality of regions.

9. A method for controlling a flexible thermoelectric device to provide dynamic thermal therapy, wherein the flexible thermoelectric device comprises a plurality of thermoelectric modules, each of which corresponds to a plurality of regions arranged sequentially, and wherein the plurality of thermoelectric modules selectively perform either exothermic or endothermic operation on the plurality of regions by control of a current, the method comprising:
controlling a supply of current to the thermoelectric modules in a first state such that a warm sensation is generated in a first region among the plurality of regions;
changing the supply of current to the thermoelectric modules to a second state different from the first state such that the warm sensation is moved to a second region adjacent to the first region; and
changing the supply of current to the thermoelectric modules to a third state from the second state, such that the warm sensation is moved to a third region adjacent to the second region,
wherein the warm sensation is moved along a direction.

10. A dynamic thermal therapy device for controlling a flexible thermoelectric element to provide dynamic thermal therapy, the dynamic thermal therapy device comprising:
the flexible thermoelectric element having a plurality of thermoelectric modules, each of which corresponds to a plurality of regions arranged sequentially, wherein the plurality of thermoelectric modules selectively perform either exothermic or endothermic operation on the plurality of regions by control of a current; and
a controller configured to:
control a supply of current to the thermoelectric modules in a first state such that a cold sensation is generated in a first region among the plurality of regions,
change the supply of current to the thermoelectric modules to a second state different from the first state such that the cold sensation is moved to a second region adjacent to the first region,
change the supply of current to the thermoelectric modules to a third state from the second state, such that the cold sensation is moved to a third region adjacent to the second region, and
wherein the controller is configured to move the cold sensation along a direction.
